(19) United States
(12) Patent Application Publication
(10) Pub. No.: US 2006/0217624 A1
(43) Pub. Date: Sep. 28, 2006

(54) System for calculating the anticipated outcome of an immediately following defibrillator shock

(75) Inventors: Helge Myklebust, Stavanger (NO); Trygve Eftestol, Forus (NO)

Correspondence Address: NIXON & VANDERHYE, PC, 901 NORTH GLEBE ROAD, 11TH FLOOR, ARLINGTON, VA, 22203, US

(73) Assignee: LAERDAL MEDICAL AS, Stavanger (NO)

(21) Appl. No.: 11/405,662

(22) Filed: Apr. 18, 2006

Related U.S. Application Data
(63) Continuation of application No. 10/070,545, filed on Jun. 4, 2002, now abandoned.

(30) Foreign Application Priority Data
Sep. 7, 1999 (NO) 1999 4344

(51) Int. Cl. A61B 5/04

(52) U.S. Cl. 600/512; 600/513; 607/5

(57) ABSTRACT

A system is provided for evaluating the probability that the outcome of an immediately following defibrillator shock will result in return of spontaneous circulation (ROSC) and for providing a decision support signal based thereon. The system includes electrodes and sensors, a module for measuring CPR and ECG related data from the electrodes and sensors, the ECG related data being measured and/or stored as ECG segments, an analysis unit connected with the module and adapted to calculate a property vector characterizing the condition of the heart from the ECG segments. The analysis unit is further adapted to calculate a probability indicator representing the probability that the outcome of an immediately following defibrillator shock will result in ROSC based on the property vector. The analysis unit is also adapted to generate a decision support signal relating to further treatment based on the property vector and/or the calculated probability indicator.

### CROSS REFERENCE TO RELATED APPLICATIONS

This application is a continuation of U.S. application Ser. No. 10/070,545, filed Jun. 4, 2002, which was the US national phase of international application PCT/NO00/00289 filed 6 Sep. 2000, which designated the US, the disclosures of which are incorporated herein by this reference.

### BACKGROUND OF THE INVENTION

1. Field of the Invention

The present invention relates to a system for calculating and using a probability indicator for the anticipated outcome of an immediately following defibrillator shock on the basis of ECG, patient information and treatment characteristics measured during sudden cardiac arrest and resuscitation.

2. Description of Related Art

Nearly 40% of all those who suffer sudden cardiac arrest could have a chance of survival if they receive good, lifesaving treatment immediately. When treatment is delayed, the chances of survival decrease, cf. the article by Holmberg S, Holmberg M: “National register of sudden cardiac arrest outside of hospitals” 1998 [1]. The treatment primarily consists of cardio-pulmonary resuscitation (CPR), which is administered until a defibrillator is in place. Thereafter, the treatment consists of alternating use of the defibrillator and CPR until resuscitation or until an ALS (ALS=“Advanced Life Support”) team arrives. The latter also includes medication and securing of the respiratory passages as part of the treatment, cf. ILCOR, “Advisory statements of the International Liaison Committee on Resuscitation.” Circulation 1997; 95:2172-2184 [6]

Scientific papers in recent years point out a number of factors that affect the chances of survival:
Time: The chance of surviving sudden cardiac arrest falls with time from heart failure until the first defibrillator shock is administered.[1]
CPR: The chance of surviving increases when someone administers CPR before the defibrillator arrives.[1]

Quality of Studies show that the quality of the CPR influences the survival.
CPR: (Cf. the publications by Wik L, Steen P A, Bircher N G. “Quality of bystander CPR influences outcome after prehospital cardiac arrest”. Resuscitation 1994; 28:195-203) [2].
   Gallagher E J, Lombardi G, Gennis P. “Effectiveness of bystander CPR and survival following out-of-hospital cardiac arrest”. 3 Am Med Assoc 1995;274:1922-5 [3] Van Hoyvegen R J, Bossaert H. “Quality and efficiency of bystander CPR”. Resuscitation 1993;26:47-52 [4])

Timing of A study shows that when the duration of sudden cardiac arrest CPR and exceeds a number of minutes, the chance of survival will defibrillator increase if the ambulance personnel first administer a period of treatment: CPR before the defibrillator is used. (Cf. Cobb L, et al. “Influence of cardiopulmonary resuscitation in patients with out-of hospital ventricular fibrillation”. JAMA, Apr. 7, 1999-Vol 281, No 13 [5]

In the case of sudden cardiac arrest, the electrical activity in the heart (ECG) will indicate the state of the heart. Today's defibrillators measure and analyse ECG in order to classify the rhythm. If the rhythm is classified as Ventricle Tachycardia (VT) or Ventricle Fibrillation (VF), defibrillator treatment may have an effect. VT is often the precursor of VF. VF will as time goes by cause the energy and oxygen reserves of the heart muscle to deplete, and eventually the rhythm will become Asystole, a rhythm characterised by very little or no electrical activity. The purpose of the defibrillator treatment is to restore the organised electrical activity of the heart and the associated blood pressure and blood circulation. This is often denoted ROSC—“Return of Spontaneous Circulation”, and is the first step towards survival.

Only a fraction of the shocks delivered actually result in ROSC. Most shocks today do not give ROSC, cf. the publications Gliner BE et al. “Treatment of out-of hospital cardiac arrest with a Low-Energy Impedance-Compensating Biphasic Waveform Automated External Defibrillator” [7], Sunde K, Eftestøl T, Askenberg C, Steen P A. “Quality evaluation of defibrillation and ALS using the registration module from the defibrillator”. Resuscitation 1999 [14]. In general, it can be said that the chance of ROSC is at its greatest immediately after sudden cardiac arrest, when the heart muscle still possesses energy reserves and oxygen. Many patients achieve ROSC after alternating use of shocks and CPR. The disadvantages of having to give many shocks are several: First of all, no CPR will be given during the shock treatment, a factor that further aggravates the situation for the vital organs, particularly for the brain. Furthermore, it has been shown that the heart muscle is also damaged by the shocks, and that the damage increases with the number of shocks and the amount of energy, cf. the publications Ewy G A, Taren D Bangert J et al. “Comparison of myocardial damage from defibrillator discharges at various dosages.” Medical instrumentation 1980; 14:9-12. [16]. For the patient, the ideal would be to be given only one shock, and for this shock to give ROSC.

Thus, for many patients, it is crucial that the administration of CPR be effective, so as to revitalise the heart through supplying a flow of blood through the heart muscle, cf. the publication Michael J R et al. “Mechanism by which augments cerebral and myocardial perfusion during cardiopulmonary resuscitation in dogs”. Circulation 1984; 69:822-835. [17]. This revitalisation can be indicated through ECG measurements, where ECG characteristics such as form, spectral flatness measurements, frequency, amplitude, energy etc. is seen to change back towards the values that would have existed immediately after the heart action was suspended, cf. the publications Eftestøl T, Aase, S O, Husøy J H. “Spectral flatness measure for characterising changes in cardiac arrhythmias”. Computers in Cardiology, [15] and Noc M, Weil M H, Gazmuri S S, Biscera I and Tang W. “Ventricular fibrillation voltage as a monitor of the effectiveness of cardiopulmonary resuscitation”. J Lab Clin Med, September 1994 [13]. This revitalisation will increase the probability of the next shock resulting in ROSC.

Unfortunately, not everyone survives. For many, the reason behind the sudden cardiac arrest is such that resuscitation is impossible. Furthermore, the time factor and the quality of the treatment will also play a part and affect the chance of survival.

Resuscitation guidelines describe a protocol that is the same for everyone, regardless of sex, race, how long the heart action has been suspended, whether a member of the public has given CPR etc. The means of resuscitation are primarily CPR and defibrillator treatment, and later also medication administered by lifesavers who have been given special training in this area. This protocol is such that if the first three shocks have no effect, CPR is to be given for 1 minute, then three more shocks, and so on. As it takes about one minute to give three shocks, the patient will be without CPR for half of the time.

Literature and other patent applications describe technology, the object of which is to guide the lifesaver in the choice between CPR and defibrillator treatment. Brown et al in U.S. Pat. Nos. 5,683,424 and 5,571,142 [10] describe a system that, based on spectral measures in VF, instructs the lifesaver to either give CPR or give a shock. A separate analysis of this method, where the method has been tested on human VF, yields results that show the method to have a low specificity, i.e. that the method will only to a limited degree reduce the number of unnecessary shocks. Noc M, Weil M H, Tang W, Sun S, Pernat A, Bisera J. “Electrocardiographic prediction of the success of cardiac resuscitation”. Crit Care Med, 1999, Vol 27, No 4 [12] describe a similar system, based on an animal model, which links the mean amplitude and dominant frequency of VF to the outcome of the defibrillator shock. Both of these methods aim to advise against defibrillator use as long as the condition of the heart is such that a shock is assumed not to have an effect, and instead use CPR. Both methods define absolute criteria based on a limited number of observations from a defined group of patients or animals.

### BRIEF SUMMARY OF THE INVENTION

The object of the present invention is to seek to constantly optimise the treatment through:
a) Sensors and electrodes connected to the patient to measure the condition of the heart, measure treatment history, and use available historical knowledge of conditions, treatment history and outcomes to calculate a probability indicator PROSC, which indicates the probability that return of spontaneous circulation (ROSC) will be the outcome of a defibrillator shock.
b) Presenting the probability indicator or using the probability indicator value in support of the decision on further treatment, for instance by considering the trend of the probability indicator.
c) Compute correlation between treatment and probability indicator, in order to identify what treatment characteristic had a positive effect on the probability indicator. Feedback this information to the user, or use this information as target values for a CPR feedback system.
d) When appropriate and practical, relay a record of each treatment (heart condition, treatment history, probability indicator and outcome data) to a centrally located computer, and use this experience to improve algorithms which calculate the probability indicator.

The object of the invention is to contribute towards giving the patient a treatment that is better suited to the individual, and which gives a greater chance of survival. The use of historical data could make it possible to adjust for individual differences and for patient group characteristics and for treatment characteristics. If such historical data were present, the system could have means of providing further input about the patient and the treatment in order to supplement information from the sensors connected to the patient:
Type of defibrillator shock and energy selection produce varying effectiveness.
Physical conditions. A big patient will receive a lower current density through the heart than a small patient, for the same energy selection.
Patient information. The system may take into account the fact that there could be a difference between men and women, based on the fact that over ⁷⁰% of those who suffer sudden cardiac arrest are men. In certain parts of the world, the duration of life has increased, so that the number of elderly who suffer sudden cardiac arrest is increasing. These may very well have received treatment over a period of time, medications, surgical procedures and aids such as pacemakers, all of which may have an effect on the PROSC.
Geography, race. The system may further take into consideration the fact that there could be differences based on lifestyle and genetic conditions, just as life expectancy varies greatly depending on geography and race.

The above could result that the calculated probability indicator had different values, depending upon patient data, patient group data or treatment data.

Using PROSC to optimise the treatment may be done in several ways. For advanced users, the most expedient will be to present the indicator graphically versus time, as a trend curve. This will immediately provide a direct indication of the state of the heart, and also indicate the effect of medication and CPR.

For groups who are not trained in relating to this type of information, the most appropriate thing will be to provide automatic decision support in the question of whether or not to give CPR, in which way CPR should be given, or whether shocks should be given. The principle of a simple decision support could be:
If the value of the indicator PROSC is less than a predefined limit, CPR is recommended. Otherwise, a defibrillator shock is recommended.
As long as CPR results in a positive trend of PROSC, continue CPR.
If CPR results in a negative trend of PROSC, the user should be directed to increase CPR efforts, alternatively to stop CPR in order to give shock.

### BRIEF DESCRIPTION OF THE DRAWINGS

The following will describe the invention in greater detail, with reference to the drawings, in which:

FIG. 1 shows system components consisting of one, alternatively several, computers in a network that communicates with a number of positioned analysis units.

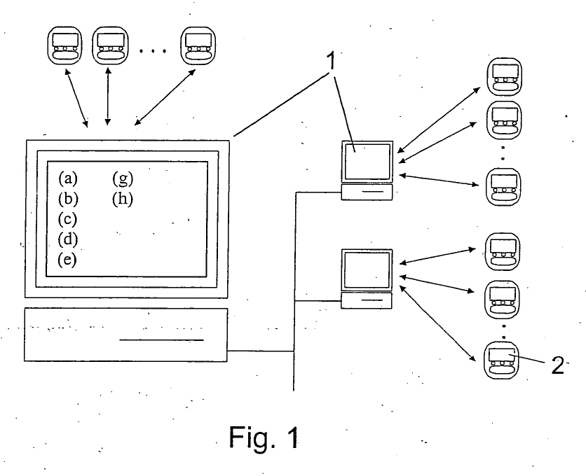

FIG. 2 shows the block diagram for a defibrillator with a built-in analysis unit.

FIG. 3 shows the elementary flow diagram for information.

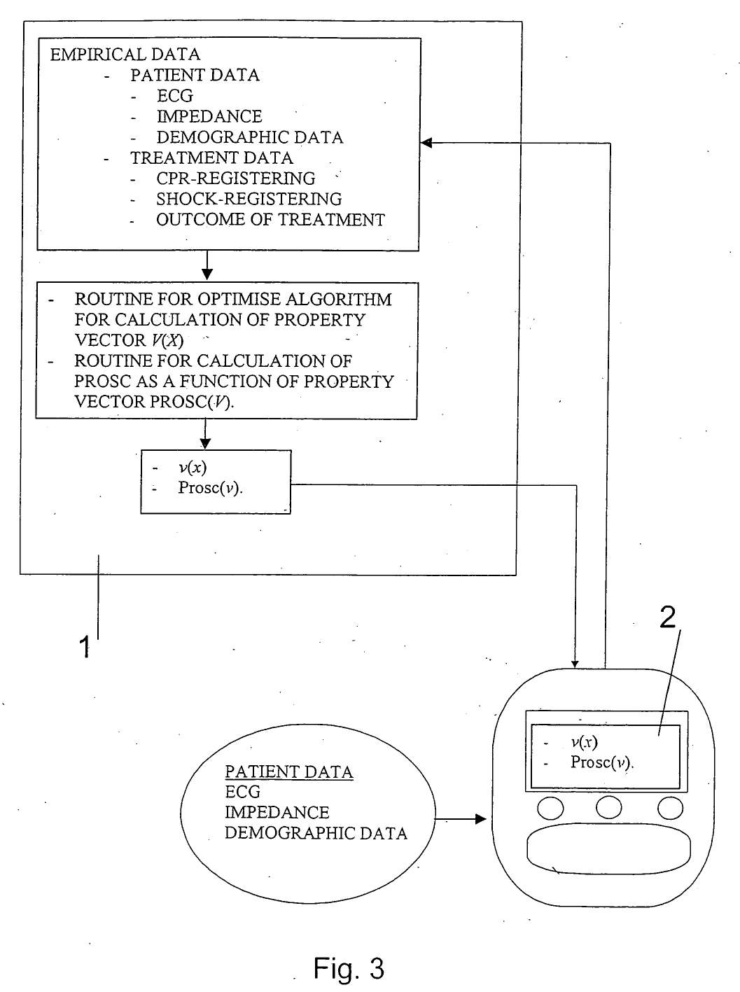

FIG. 4 shows an apparatus with electrodes connected to the patient's chest, in positions on the chest that are normally used for delivery of a defibrillator shock, as well as for measuring ECG in accordance with standard derivation II.

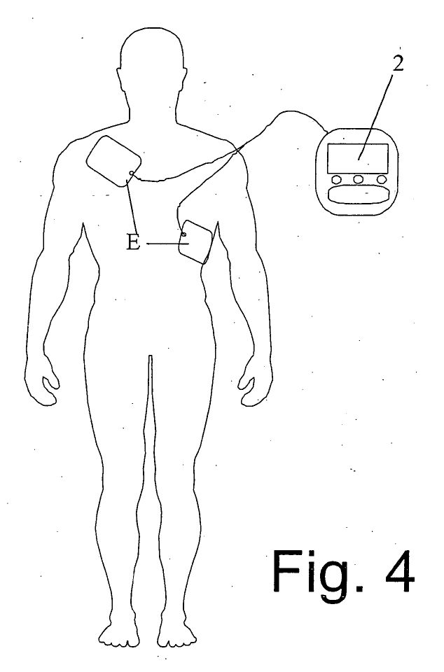

FIG. 5 shows a flow diagram for development of coefficients to optimal filter.

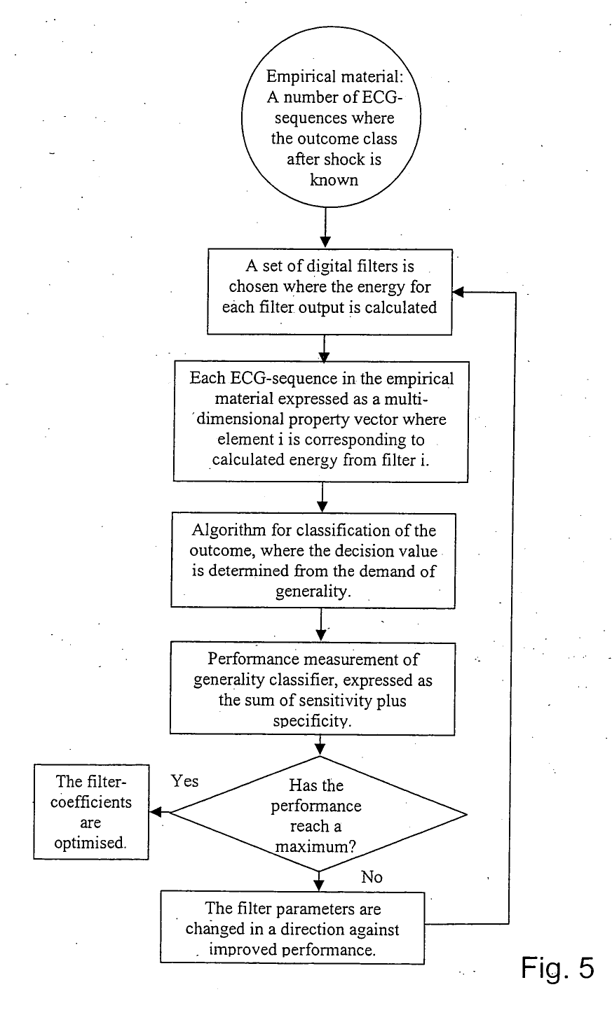

FIG. 6 shows a flow diagram for development of a classification that fulfils the requirement of generality.

FIG. 7 shows a general block diagram of the invention with focus on the analysis unit.

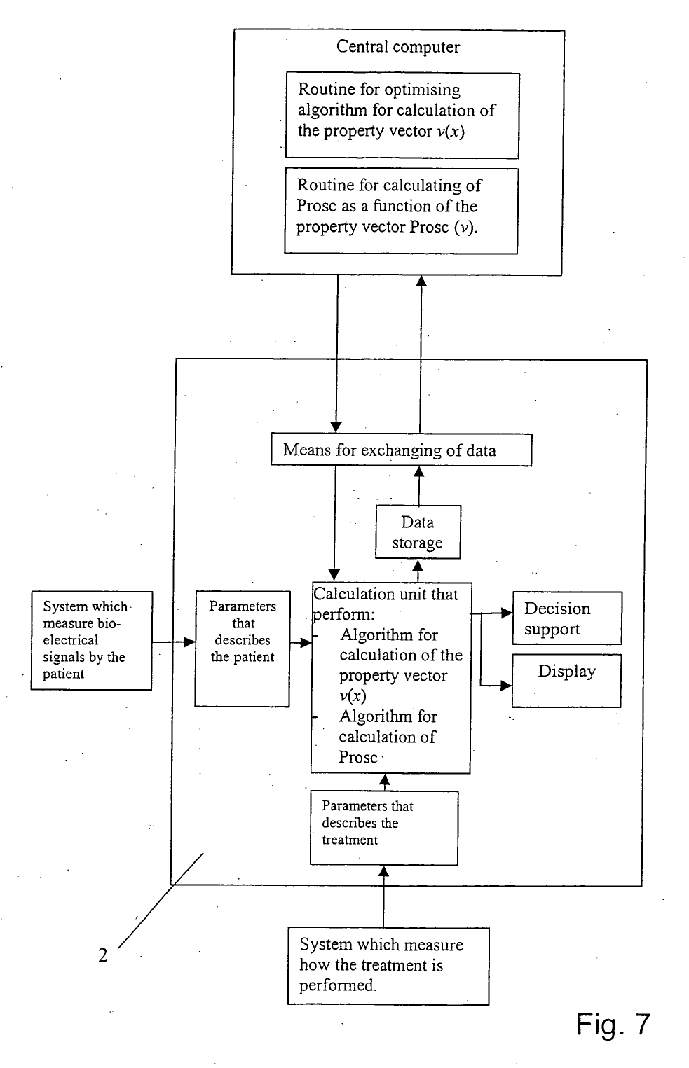

### DETAILED DESCRIPTION OF THE INVENTION

The system consists of one, alternatively several, computer(s) 1 in a network that can communicate with a number of positioned analysis units 2. These may either be integrated into equipment (U1, U2 . . . ) such as defibrillators or ECG monitors, or they may occur in or as a support product used during the resuscitation attempt. The analysis units 2 generally operate independently of the computers 1, however after use, the analysis units could deliver field data to the computer 1, and could also receive adjusted algorithms for calculation of property vector and/or PROSC

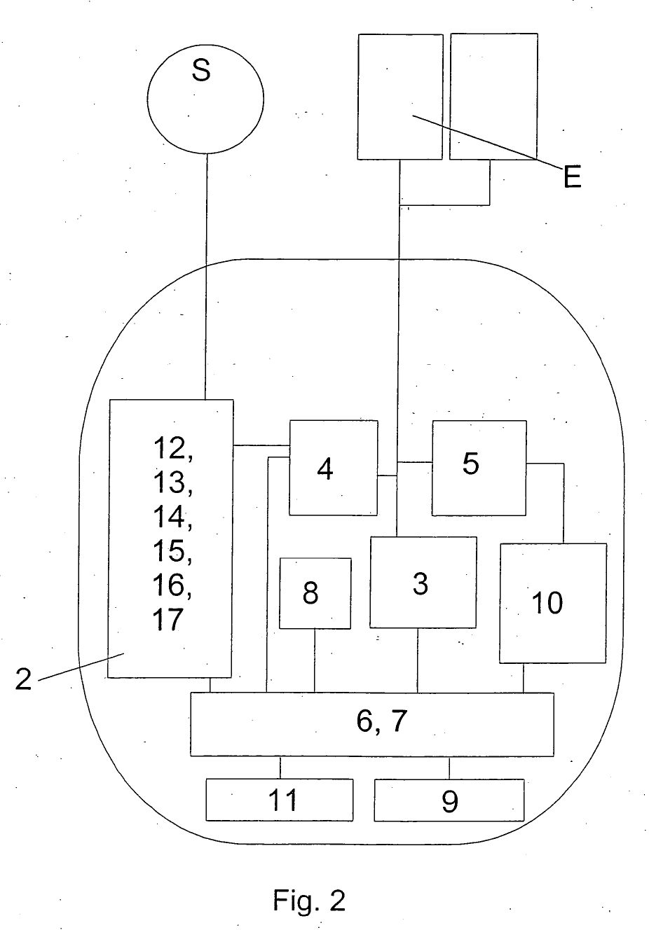

The analysis unit 2 is normally connected to other subsystems, cf. FIG. 2:

Some of these subsystems are standard in equipment such as defibrillators and ECG monitors, and these are as follows:

Electrodes E, which provide input on ECG and impedance as well as means for providing defibrillator energy to the heart, are connected to: An ECG measurement system 3, an impedance measurement system 4, the main function of which is to check if the electrodes are connected to the patient, and circuitry for high voltage generation and shock delivery 5. Further subsystems are: Processing means 6 which can classify the present ECG rhythm as shockable or non-shockable, processing means 7 which is typically a microcontroller with software, memory 8, user interface 9, power supply and battery 10, and communication means 11. Subsystems 3-11 are standard equipment in defibrillators /monitors, and will therefore not be described further in this specification.

Analysis unit 2 could be a standalone subsystem which is connected to sensors S, electrodes E and with means of receiving specific information relating to patient and treatment and having means of communicating the computed property vector and/or probability indicator. The analysis unit could also be integrated with existing input/output, signal analysis instrumentation and processing means, for instance within a defibrillator.

Analysis Unit 2 Includes the Following Units:

Unit 12 for determining one or more properties of the heart that are processed to a property vector and based on this calculate the probability of ROSC, PROSC indicator, for the patient who is connected up. Module 13, if present, for determining the blood flow through the heart, based on the measured impedance and the change of the impedance between the electrodes as a function of the pumping action of the heart and the expansion of the lungs. Module 14 for registering CPR characteristics from chest compression data, e.g. chest compression depth and rate, and ventilation data from sensors S. Module 15 for inputting patient specific information. Module 16 for inputting any medication administered; and a module 17 for correlating positive changes in PROSC or the property vector with information regarding the treatment given, and display or use this information to guide the treatment.

Further Detailed Description of the Analysis Unit 2.

Module 12, comprising an algorithm v(x) for the calculation of a property vector (v) and algorithm for calculating the probability of ROSC, PROSC, as a function of ECG from the patient who is connected up, and further as a function of specific information regarding patient and treatment:
v(x) is a set of calculations, which combined form a property vector (v). The calculation can be a set of energy calculation within determined frequency bands (optimised filters) or a set of parameters diverted from effect density spectrum, or time domain features or a combination of this.
The algorithm for the calculation of PROSC is typically an algorithm for selecting a PROSC value from a matrix (lookup table), where the reasoning for lookup is a determined by the value of the property vector, available patient information and available treatment information.

Module 13 for calculating blood flow through the heart based on the measured impedance and the change of the impedance between the electrodes as a function of the pumping action of the heart and the expansion of the lungs:
The value of the measured impedance, Zo, measured by means of an approximately constant alternating current, informs the analysis unit 2 of the impedance between the electrodes, and can be used to replace system 4.
The impedance change between the electrodes will be proportional to the change in the set of air in the lungs plus the amount of blood pumping trough the heart. The change due to air dominates. By looking at the signal between two ventilations, or by first filtering out the ventilation, it will be possible to estimate the amount of blood on the basis of the formula

$$Δ V = \frac{Δ Z \cdot ρ {L}^{2}}{Z {o}^{2}}$$

This formula is universally known, and is used in Impedance Cardiography.
ΔZ is the impedance change, p is the resistivity of the blood, L is the distance between the electrodes, and Zo is the numerical value of the impedance. A simplification of this formula is:

$$Δ V = \frac{Δ Z \cdot k}{Z {o}^{2}}$$

Here, k is a constant. This measurement will indicate to what degree the blood is flowing, and will contribute towards characterising the condition of the heart in VF/VT, This measurement serve as an indicator of ROSC in case of a successful defibrillator shock.

Module 14 for measuring and registering CPR parameters. Relevant CPR parameters are:
Inflation time and inflation volume are measured by looking at the impedance change between the electrodes. This change is several times greater than the change that takes place as a function of the blood stream from the heart, and is proportional to the amount of air in the lungs. The principle is known from other diagnostic equipment.
Compression depth calculated on the basis of signals from an accelerometer placed at the compression point.
Time between inflation and chest compression, and time between chest compression and inflation.
Proportion of CPR relative to the total treatment time
Amount of compression the sum of the product between the duration and depth of the compression

Module 15 for indicating patient specific information. This information can be passed to the analysis unit 1 e.g. by dedicated push buttons or it may come in from an external source such as a patient database or a patient journal on a PC/handheld computer. Relevant information is:

Geographical area

Age

Sex

Weight

Race

Module 16 for indicating medication and dosage given. This information can be passed on to the analysis unit 1 through dedicated push buttons, from a patient journal on a PC or other devices that log the use of medication. Relevant medicines are

Epinephrine

Lidocaine

Bretylium

Magnesium sulphate

Procainamide

Vasopressins

Thrombolysis medication

Module 17 for correlating changes in PROSC with information regarding the treatment given, and displaying or using this information to guide the treatment.
The system identifies and registers periods of PROSC with positive change. At the same time, the system identifies and registers the average of each CPR parameter measured for a period of time prior to the change and during the change, and if applicable, what medication was given during the same period.
This information can be displayed on the defibrillator screen, or it may be used to produce voice messages that guide the user to deliver CPR with parameters that are associated with a positive change in PROSC.
This information will also be of great importance to research, with a view to optimising the guidelines for CPR treatment and training.

In this regard, a principle of this invention is that there is an opportunity to improve the algorithms for the calculation of the property vector (v) and the algorithms for the calculation of the probability indicator PROSC. These algorithms are improved as a function of experience data. Experience data will typically come from a number of uses from a number of different analysis units. The experience data is then communicated from the analysis units to a central computer, which calculates improved algorithms and then communicate the improved algorithms back to the analysis units. The interval at which this is done can vary.

The computer 1 includes the following subsystems:

(a) Hardware, (b) operating system, (c) software and interface for communication in a network (d) database for field data, (e) algorithm for calculation of a property vector, (f) algorithm for calculation of PROSC, (g) algorithm for correlating changes in PROSC with information regarding patient and treatment, and (h) system for delivery and receipt of data from positioned defibrillators.

With regard to the computers, the subsystems of hardware, operating systems, software and interface are of a generic nature, and will not be described in greater detail.

Specific information about computer 1:

(d) The database for field data consists of a large amount of patient lo episode data, and contains:

Patient information: Sex, age, weight, race, medical record etc.

Geographical information

Information regarding each defibrillator shock: Curve shape, energy, timing

versus VF.

For each shock:

Preshock ECG

Preshock CPR data

Preshock medication data

Preshock impedance data

Postshock ECG

Postshock impedance data

Annotation of ROSC/No-ROSC, with outcome rhythm for each shock

(e) The algorithm for calculation of the property vector (v) makes use of mathematical methods in order to characterise the condition of the heart based on a recording of a bio-medical signal (x). The bio-medical signal is preferably ECG.

The algorithm for calculation of the property vector is hereafter denoted as v(x). v(x), which is used on empirical ECG data, provides two sets of property vectors:

A set, V1, containing n1 property vectors where the outcome of the shock is ROSC, and a set, V2, containing n2 property vectors where the outcome of the shock is no-ROSC.

In general, v(x) is defined as an operator that operates on an ECG segment, x, consisting of N samples, which generates a property vector, v, consisting of M vector elements that ideally takes care of the information in x lo that separates the group of x that results in ROSC, X1, from the group of x that results in no-ROSC, X2. The methods of property extraction are innumerable, and the literature describes some of these, which can be roughly divided into time and transform domain methods, where the object is to structure x in a manner that is appropriate for property extraction. Among preferred time domain methods are:
1. Optimised digital filters determined by L filter parameters that divides x into M channels. The energy from each of these channels is calculated, so as to make the property vector consist of M elements. These types of filters are described inter alia by T. Randen “Filter and Filter Bank Design for Image Texture Recognition” in a thesis of NTNU, October 1997 where the filters are optimised in order to achieve the best possible recognition of the different textures. For the present purpose the optimised filters are found by using a numerical gradient search algorithm (T. Coleman, M. A. Branch and A. Grace, Optimization Toolbox for Use with MATLAB, The Math Works Inc, 1999) to achieve the best possible separation of the ROSC group from the no-ROSC group. Separation ability is measured by the sum of sensitivity (degree of correct recognition of ROSC) and specified (degree of correct recognition of no-ROSC) This performance is measured by a given iteration in optimisation, and the set of parameters, which define the filters, is adjusted in the direction corresponding the increase in performance. This procedure is repeated until maximum performance is reached.
2. Selecting segments of ECG with length n, and for each segment calculate time domain variables like mean amplitude, median amplitude, number of zero crossings, mean or median slope of the rising or falling signal element.

Among preferred methods for transform domain property extractions are:
1. wavelet analysis
2. Spectral measures that are calculated on the basis of the estimate of the power density spectrum (PSD) of x. The PSD can be estimated through use of Fourier transforms. Based on the PSD, a number of features can be calculated: Frequency by the centre of gravity, frequency by the maximum point, spectral flatness measurements, and spectral energy

The relation between V1 and X1, V2 and X2 respectively are as follows: X1 containing a set of n1 ECG segments, which, when used on v(x), provides a number of property vectors V1, which all belong to the outcome class ROSC (w1). X2 containing a set of n2 ECG segments, which, when used on v(x), provides a number of property vectors V1, which all belong to the outcome class no-ROSC (w2).

(f) A system for calculation of the PROSC function is based on pattern recognition theory, and forms the second element of the classification system. In this context, the term classes is defined as the collection of measurements of the condition of the heart that corresponds to
ROSC (w1)
no-ROSC (w2)

The property vectors of the two classes are statistically described by
P(wi), i=1,2, which is the a priori probability of the two classes, i.e., before a measurement is made, the probability of one or the other outcome is known through the respective a priori probabilities.
p(v/wi) are the class specific probability density functions. These express how the measurements within the given classes are distributed. p(v) expresses the compound probability density function for the measurements, and is given by adding up the class specific probability density functions weighted by the associated a posteriori probabilities.
P(wi/v) are the a posteriori probability functions for the two classes. These functions express the probability of a given measurement belonging to wi. Bayes formula expresses P(wi/v) as a function of the above probability functions.
   P(wi|v)=P(wi)*p(v|wi)/(p(w1)+P(w2)*p(v|w2))
The sum of the a posteriori probabilities for a given vis always 1.

In the case of a given measurement, v, one wishes to determine the class allocation w1 or w2. It has been proven that the expected probability of misclassification is minimised by selecting the wi that corresponds to the maximum P(wi/v). It is further possible to define (make an estimated choice of) the cost of all types of misclassification, such that the expected risk of a given misclassification is given by the product of the cost and the a posteriori probability of the true class. The expected risk of misclassification can then be minimised by classification is a class corresponding the product with the smallest value.

In most cases, the statistics of the property vector are not known. These quantities must then be estimated before PROSC(v) can be produced. The pattern recognition theory describes a multitude of methods for this, which are based on measurements (practice data) that are examples from the various wi. Some examples:
Histogram techniques, which divides the outcome space into hypercubes in which the probabilities within each of these are calculated on the basis of the number of occurrences of the different classes within the given hypercube. This corresponds to the method used herein. In the following how statistic quantity is estimated is described.

We will start defining the quantities:
n=total number of observations in the empirical material.
n1=total number of observations corresponding ROSC outcome.
n2=total number of observations corresponding no-ROSC outcome.
nj1=total number of observations corresponding ROSC outcome within hypercube no. j.
nj2=total number of observations corresponding no-ROSC outcome within hypercube no. j

We have n=n1+n2. Estimate for a priori probability will then be
ˆP(wi)=ni/n, i=1,2.

The local estimates (within hypercube j) for the class specific probability function will then be
ˆp(v|wi)=nji/ni, i=1,2.

The local estimates for a posteriori probabilities is calculated in respect of the Bayes formula inserted estimate for a priori probability and the local class specific probability density functions. See R. J. Schalkoff. Pattern recognition: Statistical, structural and neural approaches. John Wiley & sons, New York (N.Y.), 1992
ˆP(wi|v)=nji/(nj1+nj2), i=1,2
Radial base functions, in which the probabilities at a given point are calculated on the basis of the contribution from surrounding practice data from the different classes. The contributions decrease with distance.
Parametric modelling, in which a mean value and dispersion for the different classes are used to produce analytical probability models.
Neural networks, learning vector quantization and nearest neighbour classification are some other central methods within the pattern recognition theory.

It is important that a given classifier be tested on a set of observations (test set) independently of the practice data (practice set), in order to check that the classifier yields the expected results, and if not, adjust the decision limits of the classifier. The demand is that there is consistency between practice and testing, that the classifier fulfils the requirement of generality (general applicability).

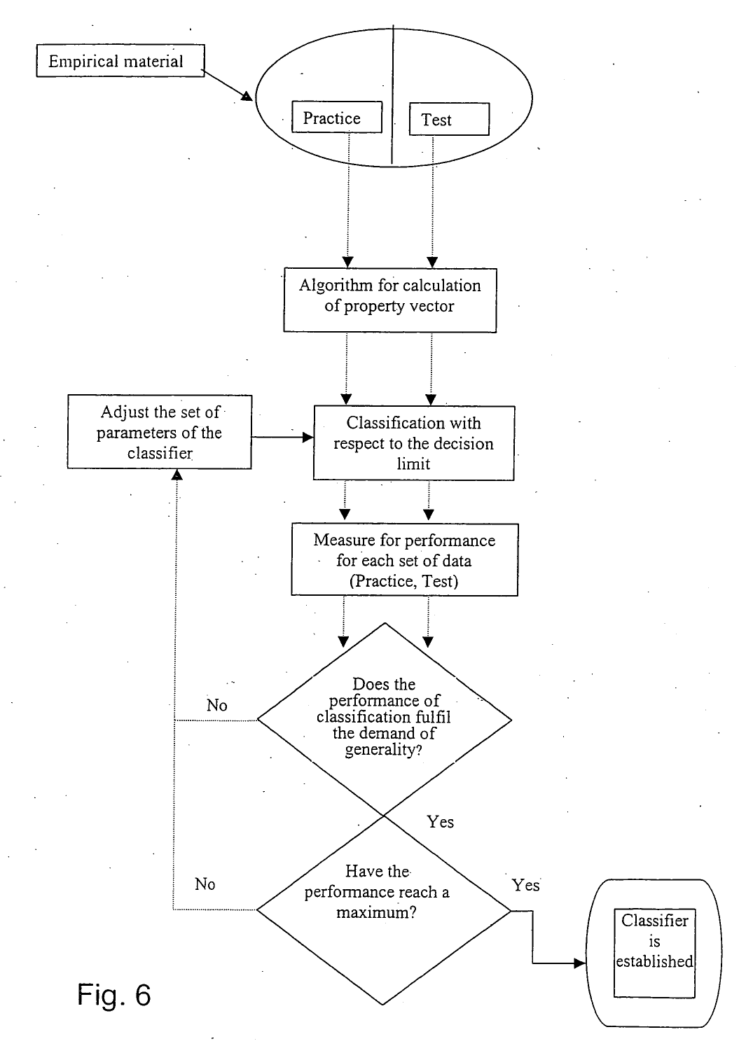

By dividing the empirical data in two parts and letting the one part represent a set of data called practice set and the other part represent a set of data called test set, the generality is defined as follows: The decision limits which, after having been used on all of the property vectors in each set of data for classification of the outcome, which provides approximately the same performance (the sum of sensitivity and specificity) for both sets of data (practice and test sets) fulfils the requirement of generality. These decision limits occur through an iterative process where the practice set is included in the calculation of the decision limit, see FIG. 6.

Those measurements v that correspond to the ROSC outcome belong in w1. The probability of a given measurement, v, belonging in w1 is given by P(w1/v). In other words, this probability function expresses the probability indicator PROSC of ROSC for a given measurement v.
PROSC(v)=P(w1|v)

As mentioned previously, different property vectors, v, can be calculated by means of a countless number of methods. Which methods and which dimension, M, is suitable for expressing PROSC(v) is assessed on the basis of the expected risk in the case of misclassification for each method. The method that minimises this risk is the most appropriate for expressing PROSC(v). FIG. 6 shows a flow diagram for an iterative development of algorithm for calculation of the property vector v. Basis for the iterative development is empirical data. As the amount of empirical data increases, this iterative process is repeated so that the ability of the property vector to predict outcome classes is increased. The iterative adjustment of the decision limit is also included so that the requirement of generality (general applicability) is fulfilled.

(g) The algorithm for correlating changes in PROSC with information regarding the patient and the treatment is mainly for scientific purposes. The defibrillator may later use the results from the correlation to guide the user during lifesaving.

PROSC(V) has been provided as described under points (d) and (e). In this analysis, ECG segments are extracted from the patient material, so that the ECG segments describe a course of treatment that is as uniform as possible. Examples of such a course of treatment may be
CPR segments
“Hands off” intervals, for instance a period for defibrillator rhythm analysis up to the shock, after a CPR period.

In these ECG segments, corresponding PROSC(v) segments are calculated as described under points (d) and (e). Consequently, the change in PROSC(v), DPROSC, is calculated for each segment. DPROSC is grouped on the basis of those treatment characteristics that are of interest with regard to the effect of the treatment. As an example, one can group DPROSC with regard to the following treatment characteristics, singly or in combination:
Different compression frequencies, compression depths, duration of chest compression
Degree of ventilation
Medication
Physiological measurements such as blood flow measurements, blood pressure etc.

Where significant differences in DPROSC occur for dissimilar treatment conditions, this information may be used to identify advantageous treatment methods. This information may be utilised through the person giving the treatment being given feedback regarding good and poor treatment.

(h) A system for delivering and receiving data from positioned analysis units. Here, no special requirements apply. The exchange of data can take place directly through use of memory modules such as PCMCIA, cordlessly by means of IR or RF communication, via networks such as the Internet, or by a direct connection between communication ports in the equipment and the computer. The most practical method these days is to have the analysis unit 2 communicate directly with the computer 1 via a local PC that it can communicate with, and to have the local computer pass the data on via the Internet.

The computer 1 contains empirical data from previous resuscitation attempts, where the outcome of the resuscitation attempt is known. The main ingredient in the empirical basis is the ECG and the associated outcome after a shock (ROSC/no-ROSC). Additional empirical data impart nuances to the relationship between outcome, treatment and patient specific factors. This additional data can be patient specific information and treatment specific information. A practical way of expressing this statistical interrelationship is through a PROSC algorithm, which is a substitute for all the empirical data, but which mathematically expresses the same relationship between the property vector and PROSC.

This algorithm is entered into the program code of the analysis unit, so that when this receives a segment of ECG, the analysis unit will first perform the same calculation of the property vector as that performed by the computer, and then use the property vector as input to the PROSC algorithm in order to calculate the probability indicator of an immediately following defibrillator shock giving ROSC. In case information about patient or therapy is available for the analysis unit, these elements can be used as input also for the PROSC algorithm.

The ever-changing forms of treatment and patient characteristics warrant a continuous update of the empirical basis. This is achieved by each analysis unit recording information about the patient, treatment and recorded ECG and CPR, and passing this on to a central computer, where the central computer repeats the grouping of the property vectors, readjusts the PROSC algorithm and passes the result back to the analysis units.

In summary, a large number of ECG segments x, from patients that has been defibrillated, and where the outcome of defibrillation is known to be either ROSC or No-ROSC, is available. These segments are grouped into either a training set or a test set. The training set is then subject to a first algorithm v(x), which computes a property vector (v) from x. The property vector may comprise a number of different properties, computed on x, either from the time domain representation of x of from the frequency domain representation. The property vector v is optimized such that vectors associated with ROSC have minimum overlap with vectors associated with No-ROSC. However, because an overlap is expected, decision regions must be chosen. There are two criteria for decision regions: The first criteria is to discriminate v associated with ROSC w1 from v associated with No-ROSC w2. The second criteria is to adjust the decision regions such that classification performance of vectors originating from the training set has about the same performance as if the vectors originated from the test set. With this criteria, generality is assured. Generality means that the risk of overtraining or over-fitting of the data is reduced. The result of this exercise means that there is an algorithm which translates the information in a segment of ECG into a property vector, and that there are decision regions defined for the classification of that property vector to either ROSC (w1) or No-ROSC (w2).

With further information, for instance information about the patient and/or the treatment, the above exercise can be repeated for each category of information. The above exercise can also be limited to have different decision regions, depending on what kind of data that is available. For instance, the decision regions can be depending on sex, race, geography, the kind of defibrillator in use, the use of drugs, and so. The probability indicator PROSC is then defined, for each value of v, as the occurrence of ROSC to the sum of the occurrence of ROSC+No-ROSC. Moreover, for each value of v, classification is made as ROSC or No-ROSC depending on the decision region. For simplification, for instance if the property vector has only got one dimension, the probability indicator can be set to just the magnitude of the vector itself.

With this in place, a therapy device, for instance a defibrillator/monitor, can now be arranged to measure ECG, input of patient information and input of treatment information. This therapy device is then arranged with the same first algorithm to translate a segment of ECG into a property vector, and a second algorithm to translate the property vector together with information on the patient and/or the treatment into a probability indicator PROSC. The use of the probability indicator can be to present the value, or trend on a display. Further, information about value and trend can be used in a third, decision support algorithm, such that recommended treatment becomes a function of both the condition of the patient and how the patient responds to the treatment. Even further use of the probability indicator is to correlate the trend of the indicator to characteristics of the treatment. When for instance a positive trend of PROSC has been identified, this trend is then correlated with CPR characteristics, and the result is, e.g., used to set target values for a CPR feedback system sent as information to the display.

As found practical and feasible, the above system can be further optimized when more data is available. For this reason, the therapy device is arranged with memory and communication means, as noted above, such that the database of information can be expanded, and the algorithms optimized.

While the invention has been described in connection with what is presently considered to be the most practical and preferred embodiment(s), it is to be understood that the invention is not to be limited to the disclosed embodiment, but on the contrary, is intended to cover various modifications and equivalent arrangements included within the spirit and scope of the appended claims.

What is claimed is:

1. A system for evaluating the probability that the outcome of an immediately following defibrillator shock performed on a patient will result in return of spontaneous circulation (ROSC) and providing a decision support signal based thereon, the system comprising:
electrodes and sensors for connection to a patient,
at least one module for measuring CPR and ECG related data from said electrodes and sensors, said ECG related data being measured and/or stored as ECG segments,
an analysis unit connected with said module,
the analysis unit being adapted to calculate, using a first algorithm, a property vector characterizing the condition of the heart from said ECG segments,
the analysis unit being adapted to calculate, using a second algorithm, a probability indicator representing the probability that the outcome of an immediately following defibrillator shock performed on the patient will result in return of spontaneous circulation (ROSC) based on said property vector, and
said analysis unit is adapted to generate a decision support signal relating to further treatment based on at least one of said property vector and said calculated probability indicator.

2. The system according to claim 1, wherein said first algorithm is chosen such that vectors computed from ECG segments associated with return of spontaneous circulation (ROSC) have minimal overlap with vectors computed from ECG segments not associated with return of spontaneous circulation (No-ROSC).

3. The system according to claim 1, wherein the property vector has at least one element, where each element is a feature computed for either the time domain representation or the frequency domain representation of the ECG segment.

4. The system according to claim 1, further comprising at least one module for receiving at least one of 1) patent information and 2) treatment information.

5. The system according to claim 4, wherein the analysis unit is adapted to calculate said probability indicator based on said probability vector and at least one of 1) patent information and 2) treatment information.

6. The system according to claim 5, wherein the second algorithm is based on empirical data, and arranged such that for each value of the property vector there is a value in a lookup table, where the reasoning for lookup is determined by the value of the property vector, available patient information, and available treatment information.

7. The system according to claim 6, further comprising a central computer arranged to collect data regarding patient treatments from said analysis unit and for computing more effective algorithms for the calculation of property vectors from ECG segments, and for computing better algorithms for the calculation of a probability of ROSC indicator, and wherein the analysis unit is connected to a data storage for storing at least one of patent information, treatment information, ECG related data and CPR related data, the analysis unit being operatively connected to said computer, and wherein the computer receives information that is stored in the data storage and the analysis unit receives optimized algorithms from the central computer for calculation of said property vectors and said probability indicator.

8. The system according to claim 1, further comprising a display, and wherein magnitude and trend of the probability indicator is presented on the display.

9. The system according to claim 1, wherein the magnitude and trend of the probability indicator is used by a decision support algorithm to generate said decision support signal.

10. The system according to claim 4, wherein a third algorithm is used to identify effective treatment, by correlating the trend and magnitude of the probability indicator with at least one of treatment information and CPR related data.

11. The system according to claim 10, wherein said identified effective treatment is presented on a display and includes at least one of depth of CPR compressions and rate of CPR compressions.

12. A system for evaluating the probability that the outcome of an immediately following defibrillator shock performed on a patient will result in return of spontaneous circulation (ROSC) and providing a decision support signal based thereon, the system comprising:
electrodes and sensors for connection to a patient,
at least one module for measuring CPR and ECG related data from said electrodes and sensors,
an analysis unit connected with said module,
the analysis unit being adapted to calculate a combination parameter characterizing the condition of the heart from said ECG related data,
the analysis unit being adapted to calculate a probability figure representing the probability that the outcome of an immediately following defibrillator shock performed on the patient will result in return of spontaneous circulation (ROSC) based on said combination parameter, and
said analysis unit is adapted to generate a decision support signal relating to further treatment based on at least one of said combination parameter and said calculated probability.

13. The system according to claim 12, further comprising at least one module for receiving at least one of 1) patent information and 2) treatment information.

14. The system according to claim 13, wherein the analysis unit is adapted to calculate said probability figure based on said combination parameter and at least one of 1) patent information and 2) treatment information.

15. The system according to claim 12, wherein the ECG related data is stored as ECG segments and a said combination parameter is calculated from at least one said ECG segment.

16. The system according to claim 12, wherein said CPR related data includes compression and ventilation data retrieved from said sensors.

17. The system according to claim 12, wherein an algorithm is provided for the analysis unit to calculate said probability figure, where the probability figure expresses the number of defibrillator shocks that results in ROSC relative the total number of defibrillator shocks for a corresponding combination of parameters, and the analysis unit has an output for the probability figure.

18. The system according to claim 12, wherein the analysis unit is connected to a data storage for storing at least one of patent information, treatment information, ECG related data and CPR related data, the analysis unit being connected to means for exchange of data, the exchange of data occurring on a regular basis to a central computer, wherein the computer receives information that is stored in the data storage and the analysis unit receives an optimized algorithm from the central computer for calculation of said probability figure.

19. The system according to claim 18, wherein the optimized algorithm is determined based on an updated set of empirical data consisting of information from a number of new patient treatments together with information from a number-of earlier performed patient treatments, which all contain ECG data segments where the outcome after subsequent shocks are known.

20. The system according to claim 12, wherein the output of the analysis unit is connected to a an external defibrillator.

21. The system according to claim 14, wherein the analysis unit identifies periods of positive change in at least one of the combination parameter and the probability figure together with parameters of the corresponding treatment, and passes this information to a receiver.

22. The system according to claim 21, wherein the receiver of said information is a display unit.

23. The system according to claim 21, wherein the analysis unit uses said information in an algorithm for generating said decision support signal.

24. A method for evaluating the probability that an immediately following defibrillator shock performed on a patient will result in return of spontaneous circulation (ROSC) and providing a decision support signal based thereon, the method comprising:
measuring CPR and ECG related data from electrodes and sensors connected to a patient,
calculating a combination parameter characterizing the condition of the heart from said ECG related data,
calculating a probability figure representing the probability that the outcome of an immediately following defibrillator shock performed on the patient will result in return of spontaneous circulation (ROSC) based on said combination parameter, and
generating a decision support signal relating to further treatment based on at least one of said combination parameter and said calculated probability figure.

25. The method according to claim 24, wherein said probability figure is calculated based on said combination parameter and at least one of 1) input patent information and 2) input treatment information.

26. The method according to claim 24, further comprising storing the ECG related data as ECG segments and a said combination parameter is calculated from each said ECG segment.

27. The method according to claim 24, further comprising providing an algorithm for calculating said probability figure, where the probability figure expresses the number of defibrillator shocks that results in ROSC relative the total number of defibrillator shocks for a corresponding combination of parameters.

28. The method according to claim 24, further comprising storing at least one of patent information, treatment information, ECG related data and CPR related data, and exchanging said stored information and data with a central computer, wherein the computer receives said stored information and data, calculates an optimized algorithm for calculation of said probability figure and transmits the optimized algorithm for use in calculating of said probability figure.

29. The method according to claim 24, further comprising identifying periods of positive change in at least one of the combination parameter and the probability figure and identifying parameters of the corresponding treatment, and passing this information to a receiver.
